(19) United States
(12) Patent Application Publication
(10) Pub. No.: US 2019/0000784 A1
(43) Pub. Date: Jan. 3, 2019

(54) PULSATILE RELEASE PHARMACEUTICAL COMPOSITION COMPRISING NAFTAZONE OR ONE OF ITS SALTS

(71) Applicant: CLEVEXEL PHARMA, Paris (FR)

(72) Inventors: Denis BRICOUT, CHENNEVIERES-SUR-MARNE (FR); Xiuping WANG ÉP. ZHANG, PARIS (FR)

(21) Appl. No.: 16/062,358

(22) PCT Filed: Dec. 14, 2016
(86) PCT No.: PCT/EP2016/081007

§ 371 Date: Jun. 14, 2018

(30) Foreign Application Priority Data
Dec. 16, 2015 (EP) 15307026.3

(51) Int. Cl. A61K 31/175; A61K 9/16; A61K 9/50

(52) U.S. Cl. CPC A61K 31/175; A61K 9/1652; A61K 9/1635; A61P 25/16; A61K 9/5073; A61K 9/5042; A61K 9/1623

(57) ABSTRACT

Disclosed is a pharmaceutical composition including naftazone or one of its pharmaceutically acceptable salts, for its use for the pulsatile release of naftazone or one of its pharmaceutically acceptable salts, wherein: a first pulse of naftazone or one of its pharmaceutically acceptable salts is released substantially immediately upon oral administration of the pharmaceutical composition; and at least one additional pulse of naftazone or one of its pharmaceutically acceptable salts is released at about 3 hours to about 8 hours following the oral administration.

The present invention concerns a pulsatile release pharmaceutical composition comprising naftazone or one of its salts. It also concerns a pharmaceutical composition comprising naftazone or one of its pharmaceutically acceptable salts, for its use for the pulsatile release of naftazone or one of its pharmaceutically acceptable salts.

The present invention also concerns the above-mentioned pharmaceutical composition for its use for treating Parkinson disease.

Naftazone is a naphtoquinone derivative which was originally registered in several European countries for treating symptoms of varicose veins and venous insufficiency, based on its venoconstrictive properties. Naftazone is currently marketed in several countries.

In particular, naftazone has been disclosed in WO01/05404 and U.S. Pat. No. 7,572,774 for treating Parkinson's disease based on its antiglutamate properties.

Parkinson's disease (PD) is a chronic, progressive neurological disease characterized by progressive impairment in motor functions that is often accompanied by disturbances in mood and cognitive functions. It affects over 3 million people worldwide. Most individuals who develop Parkinson's disease are 60 years of age or older.

Most of the treatments aim to restore dopamine signaling and thereby reduce the severity of the motor symptoms. Levodopa remains the gold-standard treatment for PD and it is frequently associated with monoamine oxidase type B (MAO-B) inhibitors, catechol-O-methyltransferase (COMT) inhibitors, inhibitors of dopamine reuptake or direct agonists of postsynaptic dopamine receptors. Although the dopamine targeted strategies address the PD related motor disturbances, they are associated with side effects. Dopamine dysregulation syndrome (DDS, including dyskinesia and “wearing-off”) and impulsive and compulsive behaviors are increasingly reported serious side-effects of dopaminergic medication, used in the treatment of PD and other disorders. Dopaminergic medication is strongly related with impulse control disorders (ICDs), while levodopa is associated with DDS. These become more severe and problematic with continued treatment and there is real unmet need to cover the limitations of dopamine replacement agents.

The biggest unmet needs are in the treatment of dyskinesia and in neuroprotection. Although patients with early disease are easier to control, a neuroprotective agent would be of benefit to all patients because of the progressive nature of the disease.

There remain key areas of unmet needs in the treatment of motor and non-motor symptoms.

The bioavailability of drugs used to treat chronic diseases such as Parkinson's disease may have important implications for their clinical utility. Drugs with low bioavailability may cause a wide variation in clinical response between patients and even in the same patient. In addition, numerous factors—including gender, age, and gastric motility—may affect a drug's bioavailability. This is especially important in patients with Parkinson's disease, who develop response fluctuations as the disease progresses.

It is common for people with Parkinson disease to take several medications, all at different doses and at different times of day, in order to manage the symptoms of the disease. While keeping track of medications can be a challenging task, understanding the medications and sticking to a schedule will provide the greatest benefit from the drugs and avoid unpleasant “off” periods due to missed doses.

Consequently, an innovative formulation and process for naftazone high dose enabling a less frequent drug administration is important to have optimal patient adherence and improve patient convenience and compliance.

The aim of the present invention is thus to provide a pharmaceutical formulation for administering a high dose of naftazone or one of its pharmaceutically acceptable salts to patients, especially to Parkinson disease patients.

The aim of the present invention is also to provide a pharmaceutical formulation for administering naftazone to Parkinson disease patients with optimal patient compliance and convenience.

Another aim of the present invention is to provide a pharmaceutical formulation containing naftazone which improves patient convenience and compliance.

Another aim of the present invention is to provide a pharmaceutical formulation containing naftazone, enabling a less frequent administration of said formulation, said formulation being preferably administered once or twice daily.

Thus, the present invention relates to a pharmaceutical composition comprising naftazone or one of its pharmaceutically acceptable salts, for its use for the pulsatile release of naftazone or one of its pharmaceutically acceptable salts, wherein:
a first pulse of naftazone or one of its pharmaceutically acceptable salts is released substantially immediately upon oral administration of said pharmaceutical composition, and
at least one additional pulse of naftazone or one of its pharmaceutically acceptable salts is released at about 3 hours to about 8 hours following said oral administration.

The present invention thus relates to a new formulation for the administration of naftazone, which is a naphtoquinone derivative, in a pulsatile manner. Such formulation may also be designated as “pulsed-release formulation” or “pulsatile delivery formulation” or “pulsatile dosage formulation”.

Within the present application, the terms “pulsatile”, “pulsed-release formulation”, “pulsatile dosage formulation”, “pulsatile delivery formulation” or “pulsatile release formulation”, is intended to represent a formulation that has the ability to release (or administer or deliver) multiple doses upon a single administration of said formulation to a patient. The individual doses can be administered at a variety of intervals, depending on the composition of said formulation.

The term “pulse”, as used herein, is intended to represent each individual temporal release of the active agent (naftazone or one of its pharmaceutically acceptable salts as defined hereafter) from the formulation to the patient.

According to the invention, the first pulse occurs substantially immediately upon oral administration of the pharmaceutical composition according to the invention such that the plasma concentration of the active agent is peaked. Then, for example, a second pulse can occur at some time after the first pulse, and said second pulse can be followed by further pulses.

Preferably, the formulation of the invention is a bi-pulsatile release pharmaceutical composition. Such bi-pulsatile formulation is able to release two doses upon a single administration of said formulation to a patient. With such formulation, the first pulse occurs substantially immediately upon oral administration of the pharmaceutical composition according to the invention and a second pulse (and last pulse) occurs at about 3 hours to about 8 hours following said oral administration.

“Pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio.

The term “pharmaceutically acceptable salt” refers to salts which retain the biological effectiveness and properties of the compounds of the invention and which are not biologically or otherwise undesirable. In many cases, the compounds of the invention are capable of forming acid and/or base salts by virtue of the presence of amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts see Berge, et al. ((1977) J. Pharm. Sd, vol. 66, 1). The expression “non-toxic pharmaceutically acceptable salts” refers to non-toxic salts formed with nontoxic, pharmaceutically acceptable inorganic or organic acids or inorganic or organic bases. For example, the salts include those derived from inorganic acids such as hydrochloric, hydrobromic, sulfuric, sulfamic, phosphoric, nitric, and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, fumaric, methanesulfonic, and toluenesulfonic acid and the like.

The present invention also relates to a pharmaceutical composition comprising:
at least one immediate-release pharmaceutical system containing naftazone or one of its pharmaceutically acceptable salts, and
at least one controlled-release pharmaceutical system containing naftazone or one of its pharmaceutically acceptable salts,

wherein said pharmaceutical composition delivers naftazone or its pharmaceutically acceptable salts to a patient in a pulsatile manner upon administration of said composition to said patient.

According to an embodiment, the pharmaceutical composition according to the invention is a pulsatile delivery formulation comprising one immediate-release pharmaceutical system and one controlled-release pharmaceutical system.

Within such embodiment, the immediate-release pharmaceutical system is intended to deliver the first pulse as described above and the controlled-release pharmaceutical system is able to deliver the second pulse as described above.

Within the present application, the controlled-release pharmaceutical system may also be referred as “modified-release pharmaceutical system”.

The present invention also relates to a bi-pulsatile release pharmaceutical composition comprising:
at least one immediate-release pharmaceutical system containing naftazone or one of its pharmaceutically acceptable salts, that provides a first pulse of naftazone or one of its pharmaceutically acceptable salts, and
at least one controlled-release pharmaceutical system containing naftazone or one of its pharmaceutically acceptable salts, that provides a second pulse of naftazone or one of its pharmaceutically acceptable salts, wherein:
the first pulse is released substantially immediately upon oral administration,
the second pulse is released at about 3 hours to about 8 hours following oral administration.

The present invention relates to a bi-pulsatile release pharmaceutical composition comprising naftazone or one of its pharmaceutically acceptable salts, for its use for the pulsatile release of naftazone or one of its pharmaceutically acceptable salts, said composition comprising:
at least one immediate-release pharmaceutical system containing naftazone or one of its pharmaceutically acceptable salts, that provides a first pulse of naftazone or one of its pharmaceutically acceptable salts, and
at least one controlled-release pharmaceutical system containing naftazone or one of its pharmaceutically acceptable salts, that provides a second pulse of naftazone or one of its pharmaceutically acceptable salts,

wherein:
the first pulse is released substantially immediately upon oral administration,
the second pulse is released at about 3 hours to about 8 hours following oral administration.

According to a preferred embodiment, the present invention relates to a bi-pulsatile release pharmaceutical composition as defined above, comprising one immediate-release (IR) pharmaceutical system as defined above, and one controlled-release (CR) pharmaceutical system as defined above.

According to an embodiment, both immediate-release and controlled-release pharmaceutical systems contain at least 40 mg of naftazone.

Preferably, the immediate-release pharmaceutical system contains from to 40 mg to 180 mg of naftazone.

Preferably, the controlled-release pharmaceutical system contains from 40 mg to 240 mg of naftazone.

According to a preferred embodiment, both immediate-release and controlled-release pharmaceutical systems contain from 40 mg to 60 mg of naftazone

Immediate-Release Pharmaceutical System

According to an embodiment, in the pharmaceutical composition according to the invention as defined above, the immediate-release pharmaceutical system is made of solid particles of naftazone or of one of its pharmaceutically acceptable salts.

Preferably, said solid particles are spherical particles. Within the present application, these spherical particles may indifferently be designated “spherical pellets” or “microspheres” or “microgranules”. These particles of the immediate-release pharmaceutical system as defined above may also be referred as “non-coated particles”.

Preferably, said solid particles are spherical particles the particle size of which being comprised between 500 μm to 1,500 μm, preferably from 800 μm to 1,250 μm, more preferably from 1,000 μm to 1,250 μm, and most preferably of about 1,000 μm.

Within the present application, the term “particle size” denotes the mean diameter of said particles. The size distribution of particles is determined using a mechanical sieve shaker (Retsch) with progressive of standards sieves between 800 μm and 1,250 μm.

According to a preferred embodiment, the spherical particles as defined above comprise from 30% to 80% of naftazone, preferably from 40% to 70%, preferably about 60%, by weight of naftazone or of one of its pharmaceutically acceptable salts, relative to the total weight of said spherical particles.

The spherical particles of the immediate-release pharmaceutical system as defined above may also comprise at least one excipient, and preferably several excipients.

According to an embodiment, the spherical particles comprise at least one excipient chosen from the group consisting of: disintegrants, fillers, diluents, plasticizers, surfactants, binders, lubricants, and mixtures thereof.

The spherical particles according to the invention may comprise at least one disintegrant chosen from the group consisting of: croscarmellose sodium, sodium starch glycolate, crospovidone, and mixtures thereof.

As disintegrants, one may also cite low substituted hydroxypropyl cellulose.

According to the invention, the preferred disintegrants are chosen from the group consisting of: croscarmellose sodium, sodium starch glycolate, and mixtures thereof.

The spherical particles according to the invention may comprise at least one filler or diluent chosen from the group consisting of: microcrystalline cellulose, sorbitol, dextrin, lactose, mannitol, cyclodextrins, carrageenan, xanthan gum, starch, sucrose, pectin and mixtures thereof.

According to the invention, the cyclodextrins are indifferently chosen from α-, β-, and γ-cyclodextrins.

The spherical particles according to the invention may comprise at least one plasticizer chosen from the group consisting of: microcrystalline cellulose, carrageenan, xanthan gum, chitosan, pectin and mixtures thereof.

Preferably, the plasticizer is carrageenan or microcrystalline cellulose.

The spherical particles according to the invention may comprise at least one lubricant chosen from the group consisting of: polyethylene glycol, propylene glycol, glycerine, and mixtures thereof.

The spherical particles according to the invention may comprise at least one surfactant chosen from the group consisting of sodium lauryl sulfate, polysorbate, and mixtures thereof.

The spherical particles according to the invention may comprise at least one binder, such as hypromellose, povidone, gelatin, starch or sucrose.

As preferred excipients according to the invention, one may cite: croscarmellose sodium, sodium starch glycolate, crospovidone, microcrystalline cellulose, sorbitol, dextrin, lactose, mannitol, cyclodextrins, carrageenan, xanthan gum, starch, sucrose, carrageenan, xanthan gum, chitosan, pectin, polyethylene glycol, propylene glycol, glycerin, sodium lauryl sulfate, polysorbate, and mixtures thereof.

Among the excipients suitable for the present invention, one may also cite hydroxypropyl cellulose or sodium alginate.

According to a preferred embodiment, the above-mentioned spherical particles comprise at least 0.05%, preferably from 0.05% to 0.25%, and more preferably from 0.05% to 0.15%, by weight of at least one surfactant, preferably of sodium lauryl sulfate, relative to the total weight of said spherical particles.

According to a preferred embodiment, the above-mentioned spherical particles comprise from 5% to 35%, and preferably from 5% to 15%, by weight of at least one plasticizer, preferably of microcrystalline cellulose, relative to the total weight of said solid particles.

According to an embodiment, the spherical particles as mentioned above comprise from 5% to 40%, preferably from 10% to 30%, by weight of carrageenan, relative to the total weight of said spherical particles.

According to an embodiment, the spherical particles as mentioned above comprise from 5% to 30%, preferably from 5% to 15%, by weight of sorbitol, relative to the total weight of said spherical particles.

According to an embodiment, the spherical particles as mentioned above comprise from 0.01% to 0.25%, preferably from 0.05% to 0.15%, by weight of sodium lauryl sulfate, relative to the total weight of said spherical particles.

Preferably, the spherical particles as mentioned above comprise:
from 30% to 80%, preferably from 40% to 70%, more preferably about 60%, by weight of naftazone or of one of its pharmaceutically acceptable salts, relative to the total weight of said spherical particles,
from 5% to 35%, preferably from 5% to 15%, by weight of microcrystalline cellulose, relative to the total weight of said spherical particles,
from 5% to 40%, preferably from 10% to 30%, by weight of carrageenan, relative to the total weight of said spherical particles, and
from 5% to 30%, preferably from 5% to 15%, by weight of sorbitol, relative to the total weight of said spherical particles; and
from 0.01% to 0.25%, preferably from 0.05% to 0.15%, by weight of sodium lauryl sulfate, relative to the total weight of said spherical particles.

Controlled-Release Pharmaceutical System

As mentioned above, the pharmaceutical composition according to the invention also comprises a controlled-release pharmaceutical system containing naftazone or one of its pharmaceutically acceptable salts as defined above.

According to an embodiment, the controlled-release pharmaceutical system is made of coated solid particles, in particular of coated spherical particles, of naftazone or of one of its pharmaceutically acceptable salts.

The solid particles of the controlled-release pharmaceutical composition according to the invention are prepared from the solid particles as mentioned above and they further comprise at least one coating layer. Preferably, the controlled-release pharmaceutical system is made of coated spherical particles of naftazone or of one of its pharmaceutically acceptable salts.

Preferably, the particle size of said coated spherical particles is comprised from 500 μm to 1,500 μm, preferably from 800 μm to 1,250 μm, and preferably of about 1,000 μm.

According to an embodiment, the coated solid particles are made of spherical particles of naftazone or of one of its pharmaceutically acceptable salts, comprising at least one coating layer, and preferably one coating layer or two coating layers.

According to a preferred embodiment, the coated solid particles are made of spherical particles of naftazone or of one of its pharmaceutically acceptable salts, comprising two coating layers.

According to the invention, the coating layers may be chosen from pH-dependent and pH-independent coatings.

Preferably, in the pharmaceutical composition according to the invention, the coated solid particles are made of spherical particles of naftazone or of one of its pharmaceutically acceptable salts, comprising at least one coating layer surrounding the spherical particles.

pH-Independent Coating:

According to an embodiment, the coated spherical particles as mentioned above comprise one first coating layer surrounding the spherical particles, and a second coating layer surrounding the first coating layer.

Preferably, the coated spherical particles according to the invention comprise a first coating layer containing at least one swelling agent chosen from the group consisting of: croscarmellose sodium, low substituted hydroxypropyl cellulose, sodium starch glycolate, crospovidone, and mixtures thereof.

Preferably, the swelling agent is low substituted hydroxypropyl cellulose.

Low substituted hydroxypropyl cellulose is also referred to “cellulose, 2-hydroxypropyl ether (low substituted)”. This excipient is well known in the art and is in particular described in EP 1 099 709 or EP 1 054 019.

Especially, compared to hydroxypropyl cellulose, low substituted hydroxypropyl cellulose has only a small proportion of the three free hydroxyl groups per glucose subunit converted to a hydroxypropyl ether. When dried at 105° C. for one hour, it contains no less than 5% and not more than 16% of hydroxypropoxy groups (—OCH₂CHOHCH₃).

According to the invention, the term “low substituted hydroxypropyl cellulose” refers to hydroxypropyl cellulose having a hydroxypropoxyl content ranging from 5% to 16% by weight.

According to the invention, the first coating layer may also comprise at least one binder chosen from the group consisting of: hypromellose, povidone, and mixtures thereof. Preferably, the binder is hypromellose.

According to the invention, the first coating layer may also comprise at least one plasticizer chosen from the group consisting of: polyethylene glycol, dibutyl sebacate, phthalate, propylene glycol, triethyl citrate and mixtures thereof.

Preferably, the plasticizer is polyethylene glycol.

According to a preferred embodiment, the coated spherical particles according to the invention comprise a first coating layer containing low substituted hydroxypropyl cellulose, hypromellose, and polyethylene glycol.

Preferably, in the first coating layer as defined above, the amount of low substituted hydroxypropyl cellulose is comprised from 5% to 40%, in particular from 10% to 30%, by weight in relation to the total weight of the above-mentioned coated solid particles.

The coated solid particles as defined above may also comprise a second coating layer. Preferably, this second coating layer is a pH-independent coating layer.

According to a preferred embodiment, the second coating layer comprises at least one hydrophobic coating agent, preferably ethylcellulose.

Preferably, the second coating layer comprises from 1% to 40%, in particular from 10% to 30%, of ethylcellulose by weight in relation to the total weight of the above-mentioned coated solid particles.

pH-Dependent Coating

Preferably, in the pharmaceutical composition according to the invention, the coated solid particles are made of spherical particles of naftazone or of one of its pharmaceutically acceptable salts, comprising at least one pH-dependent coating layer surrounding the spherical particles.

According to an embodiment, the coated spherical particles as mentioned above comprise one coating layer pH-dependent surrounding the spherical particles.

According to an embodiment, the coated spherical particles according to the invention comprise one coating layer containing at least one anionic copolymer chosen from the group consisting of: methacrylic acid and an ester chosen from the group consisting of: methyl methacrylate, ethyl acrylate, methyl acrylate, and mixtures thereof.

According to an embodiment, the coated spherical particles according to the invention comprise one coating layer containing at least one anionic copolymer chosen from the group consisting of: methacrylic acid and ethyl acrylate or methyl methacrylate.

Preferably, the coated spherical particles according to the invention comprise one coating layer containing at least one anionic copolymer chosen from the group consisting of: methacrylic acid and ethyl acrylate or methyl methacrylate, with a proportion of 50% by weight ethyl acrylate or methyl methacrylate and 50% by weight methacrylic acid (EUDRAGIT® L, EUDRAGIT® L 100-55, Acryl-EZE® or EUDRAGIT® L 100 types) and mixtures thereof. In the intestinal medium or simulated intestinal fluid, the release can be started at selected pH between pH 5.5 to pH 7.

According to an embodiment, the coated spherical particles according to the invention comprise one coating layer containing at least one anionic copolymer chosen from the group consisting of: methacrylic acid and methyl methacrylate.

Preferably, the coated spherical particles according to the invention comprise one coating layer containing at least one anionic copolymer chosen from the group consisting of: methacrylic acid and methyl methacrylate, with a proportion of 20 to 40% by weight methacrylic acid and 80 to 60% by weight methyl methacrylate (EUDRAGIT® S type).

According to an embodiment, the coated spherical particles according to the invention comprise one coating layer containing at least one anionic copolymer chosen from the group consisting of: methyl methacrylate, methyl acrylate and methacrylic acid.

Suitable methacrylate copolymers are those consisting of 10 to 30% by weight methyl methacrylate, 50 to 70% by weight methyl acrylate and 5 to 15% by weight methacrylic acid (EUDRAGIT® FS type). EUDRAGIT® FS is a copolymer polymerized out of 25% by weight methyl methacrylate, 65% by weight methyl acrylate and 10% by weight methacrylic acid. EUDRAGIT® FS 30 D is a dispersion comprising 30% by weight EUDRAGIT® FS. In the intestinal medium or simulated intestinal fluid, the release can be started at pH 7.2.

Preferably, the coated spherical particles according to the invention comprise one coating layer containing at least one anionic copolymer chosen from the group consisting of: mixture of EUDRAGIT® L type and EUDRAGIT® S type with different ratio to obtain adequate release at pH between pH 6.0 and pH 7.2.

Preferably, the copolymer agent is Eudragit®FS 30D.

According to the invention, the coating layer may also comprise at least one plasticizer, preferably chosen from the group consisting of: polyethylene glycol, dibutyl sebacate, phthalate, Polysorbate 80, triethyl citrate, PlasACRYL™ T20 and mixtures thereof. Preferably, the plasticizer PlasACRYL™ T20.

According to the invention, the coating layer may also comprise at least one anti tacking or glidant agent, preferably chosen from the group consisting of: talc, PlasACRYL™ T20 and mixtures thereof. Preferably, the anti-tacking or glidant is PlasACRYL™ T20.

According to a preferred embodiment, the coated spherical particles according to the invention comprise one coating layer containing Eudragit®FS 30D and PlasACRYL™ T20.

Preferably, in the pH-dependent coating layer as defined above, the amount of Eudragit®FS 30D is comprised from 5% to 40%, in particular from 10% to 25%, by weight in relation to the total weight of the above-mentioned coated solid particles.

The coating layer may be prepared by fluid bed laboratory system with Wuster container. The pH-dependent coating layer is preferably Eudragit®FS 30D with in particular between 10% and 25% of weight gain.

The pharmaceutical composition according to the invention may be used in the form of a capsule or a tablet. In particular, the immediate-release and controlled-release pharmaceutical systems may be filled in capsules at desired proportions or compressed to tablets.

In particular, the pharmaceutical composition according to the invention is in the form of a capsule comprising a mixture of immediate-release and controlled-release pharmaceutical systems, that is to say a mixture of coated particles and non-coated particles as defined above.

The pharmaceutical compositions of the present invention may be prepared according to the following process:

1) preparation of the non-coated spherical particles (corresponding to the immediate-release system);

2) coating of the spherical particles from step 1) for obtaining coated spherical particles; and

3) filling capsules with the required amount of spherical particles from step 1) and 2), respectively.

The first step as mentioned above may be carried out as explained below.

The spherical particles may be prepared by wet granulation followed by extrusion-spheronization. The active substance and the excipients such as in particular a disintegrant, a filler/diluent and a surfactant, may be blended together in a high-shear granulator, and granulated to form agglomerates by adding/spraying a granulating fluid such as water. The wet mass can be extruded and spheronized to produce spherical particles (pellets).

The second step as mentioned above may be carried out as explained below for pH-independent coating.

The first coating layer may be prepared by fluid bed laboratory system with Wuster container. The first coating layer is preferably a swelling layer with disintegrant with in particular between 10% and 30% of weight gain.

The second coating layer may be prepared by fluid bed laboratory system with Wuster container. The second coating layer is preferably a hydrophobic layer with ethylcellulose with in particular between 10% and 30% of weight gain.

The second step as mentioned above may be carried out as explained below for pH-dependent coating.

The coating layer may be prepared by fluid bed laboratory system with Wuster container. The pH-dependent coating layer is preferably Eudragit®FS 30D with in particular between 10% and 25% of weight gain.

The present invention also relates to the pharmaceutical composition as defined above, for its use for the treatment of Parkinson disease.

In the context of the invention, the term “treating” or “treatment”, as used herein, means alleviating, inhibiting the progress of, or preventing the disorder or condition to which such term applies, or one or more symptoms of such disorder or condition.

According to a preferred embodiment, the pharmaceutical composition according to the invention is administered once or twice a day.

### DESCRIPTION OF FIGURES

FIG. 1 represents the results of the dissolution test of microspheres of uncoated naftazone (example 1). The graph represents the % of release of naftazone over time (in hours).

FIG. 2 represents the results of the dissolution test of microspheres of coated naftazone (example 2). The graph represents the % of release of naftazone over time (in hours).

The graph with squares corresponds to microspheres with L-HPC coating at 16% and Surelease at 15% and the graph with triangles corresponds to microspheres with L-HPC coating at 19% and Surelease at 22%.

FIG. 3 represents the results of the dissolution test of the mixture of microspheres of uncoated naftazone and microspheres of coated naftazone (example 3). The graph represents the % of release of naftazone over time (in hours).

The graph with dotted line corresponds to the mixture of microspheres wherein CR microspheres are made with L-HPC coating at 16% and Surelease at 15% and the graph with straight line corresponds to the mixture of microspheres wherein CR microspheres are made with L-HPC coating at 19% and Surelease at 22%.

FIG. 4 represents the results of the dissolution test of naftazone microspheres of pH-dependent coated naftazone. There is no dissolution occurred in pH 6.8 medium. Once the microspheres transferred to pH 7.3 medium, the dissolution of naftazone occurs rapidly (example 4). The graph represents the % of release of naftazone over time (in hours).

FIG. 5 represents the results of the dissolution test of naftazone microspheres with only one coating layer of Surelease (example 5). The graph represents the % of release of naftazone over time (in hours).

FIG. 6 represents the dissolution profiles of naftazone capsule IR vs. SR based on Compritol (example 6). The graph represents the % of release of naftazone over time (in hours).

The graph with circles corresponds to the capsule IR; the graph with dotted line and triangles corresponds to SR formulation using grain at size of 300 μm comprising naftazone and Compritol at 1/1 ratio; the graph with straight line and triangles corresponds to SR formulation with grain at size of 300 μm comprising naftazone and Compritol at 1/2 ratio; the graph with straight line and triangles corresponds to SR formulation with grain at size of 800 μm comprising naftazone and Compritol at 1/1 ratio; the graph with dotted line and triangles corresponds to SR formulation with grain at size of 800 μm comprising naftazone and Compritol at 1/2 ratio.

FIG. 7 represents mean normalized plasma concentration-versus-time-profiles and PK parameters for naftazone after single oral administration of F1, F2 and F3 (example 6). The graph represents the normalized plasma concentrations (ng/mL) over time (in hours). The graph with circles corresponds to F1, the graph with squares corresponds to F2, and the graph with triangles corresponds to F3.

### EXAMPLES

### Example 1: Preparation of Uncoated Naftazone Microspheres (IR)

Microspheres are prepared from the following ingredients:

| Ingredients | Amount (g) |
|---|---|
| Naftazone | 60 |
| Carrageenan, NF/EP (Gelcarin PH 911) | 20 |
| Microcrystalline Cellulose, NF/EP | 10 |
| (Avicel PH 101) | |
| Sorbitol, NF/EP (Neosorb P 100T) | 9.9 |
| Sodium lauryl sulfate, EP | 0.1 |
| Total | 100 |

Purified water is also used and removed during the preparation process.

Naftazone, Carregeenan, microcrystalline cellulose and sorbitol are dry blended in a Turbula mixer during 15 mn at 23 rpm. A granulation solution is prepared at 1% with sodium lauryl sulfate and purified water. Request quantity of sodium lauryl sulfate 1% is adapted to the granulation volume. Dry blend is transferred in a Glatt granulator and the granulation solution is added. Granulation is performed at low shear 50 rpm condition.

The resulting granulated product is extruded (die plate hole 1 mm) and spheronized in a Caleva extruder/spheronizer. Extrusion speed is performed at 50 rpm and spheronisation speed between 1,800 rpm and 2,300 rpm during seven minutes to obtain spherical and homogenous microspheres. Finally microspheres are tray dried at a temperature not beyond 40° C. The residual moisture of microspheres is preferably at about 1% to 2% w/w.

Dissolution Test

In order to estimate the characteristic of naftazone release of prepared microspheres described in example 1, a dissolution test was performed under following condition:

| | |
|---|---|
| Apparatus | Apparatus 1 (basket) conforming to |
| | Pharmacopeias (for example USP <711>) |
| Dissolution Medium | SDS solution 1% in water |
| Dissolution volume | 1,000 mL |
| Basket rotation speed | 75 rpm |
| Sampling time point | 5, 10, 15, 30, 60, 120, 240 and 480 min |
| Bath temperature | 37.0° C. (±0.5° C.) |

Dissolution Analysis Method
Using a graduated cylinder, measure and introduce 1000 mL of dissolution medium into each dissolution vessel
Equilibrate filled vessels to 37° C.±0.5° C.
Introduce the basket containing the quantity of uncoated microspheres corresponding to equivalent of 40 mg naftazone into dissolution vessel and stir at 75 rpm
At each sampling time, withdraw about 2 mL of solution from each vessel, filter and dilute with sample solvent (water/acetonitrile, 1/1:v/v)
Analyze diluted samples using HPLC/UV method

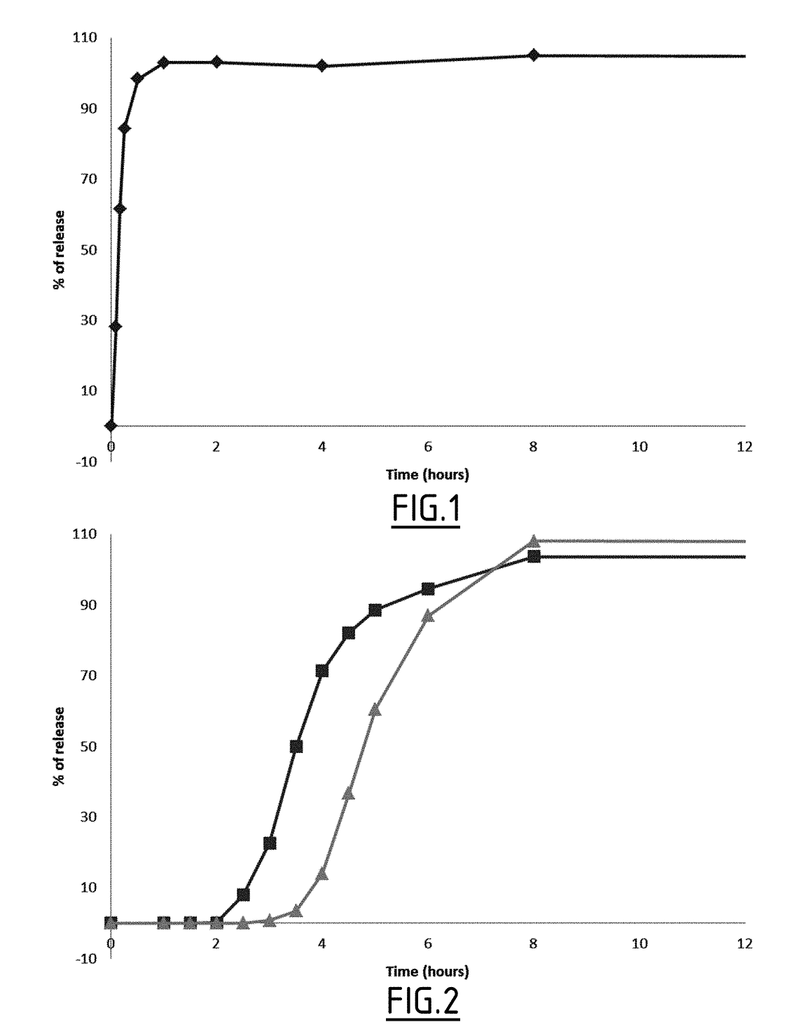

As shown in FIG. 1 concerning the results of the dissolution test of the IR microspheres, uncoated naftazone microspheres show a very fast dissolution completed within 30 or 60 minutes.

### Example 2: Preparation of Controlled-Release Naftazone Microspheres (CR) with pH-Independent Coating Layer

Starting from the microspheres of example 1, a first coating is carried out by using the first layer (swelling layer) containing:

| Ingredients | Amount (g) |
|---|---|
| Low substituted hydroxypropyl cellulose NF/JP | 6 |
| (L-HPC grade LH 31) | |
| Hypromellose (Methocel E5 Premium LV | 1 |
| USP/EP/JP) | |
| PEG 6000 USP/NF | 0.6 |
| Purified water (removed during processing) | 92.4 |
| Total | 100 |

100 g of naftazone microspheres are placed into a fluidized bed (Mini Glatt) with wurster and pre-heated to 41° C. During processing, coating conditions are maintained for temperature product (38° C.), spray rate (2 g/min), air flow (20 m³/h) and spray pressure (1 b).

Then, a second coating is carried out. The second layer (controlled layer) with a pH independent layer which contains: Surelease (commercially ethylcellulose aqueous dispersion), and water.

Surelease 25% is diluted to 15% with purified water. 100 g of first layer coated naftazone microspheres are placed into a fluidized bed (Mini Glatt) with wurster and pre-heated to 42° C. During processing of second layer, coating conditions are maintained for temperature product (40° C.), spray rate (1.8 g/min), air flow (24 m³/h) and spray pressure (1.2 bar). After coating, the microspheres are heat cured at 60° C. for two hours to form complete film.

Dissolution Test

In order to estimate the characteristic of naftazone release of coated microspheres described in example 2, a dissolution test was performed under following condition:

| | |
|---|---|
| Apparatus | Apparatus 1 (basket) conforming to |
| | Pharmacopeias (for example USP <711>) |
| Dissolution Medium | SDS solution 1% in water |
| Dissolution volume | 1,000 mL |
| Basket rotation speed | 75 rpm |
| Sampling time point | 1, 1.5, 2, 2.5, 3, 3.5, 4, 4.5, 5, 6, 8 and 24 h |
| Bath temperature | 37.0° C. (±0.5° C.) |

Dissolution Analysis Method
Using a graduated cylinder, measure and introduce 1,000 mL of dissolution medium into each dissolution vessel
Equilibrate filled vessels to 37° C.±0.5° C.
Introduce the basket containing the quantity of coated microspheres corresponding to equivalent of 40 mg naftazone into dissolution vessel and stir at 75 rpm
At each sampling time, withdraw about 2 mL of solution from each vessel, filter and dilute with sample solvent (water/acetonitrile, 1/1:v/v)
Analyze diluted samples using HPLC/UV method

As shown in FIG. 2 concerning the results of the dissolution test of the CR microspheres, with 22% of Surelease as the controlled layer, the lag time of delayed release is close to 4 hours, whereas 15% of Surelease decreases the lag time release to 2 hours. The slope of dissolution curves is similar using 16% or 19% of L-HPC coating.

### Example 3: Mixture of Microspheres of Examples 1 and 2

A mixture of IR microspheres (example 1) and of CR microspheres (example 2) is prepared.

The mixture is such that both IR microspheres and CR microspheres contain the same amount of naftazone.

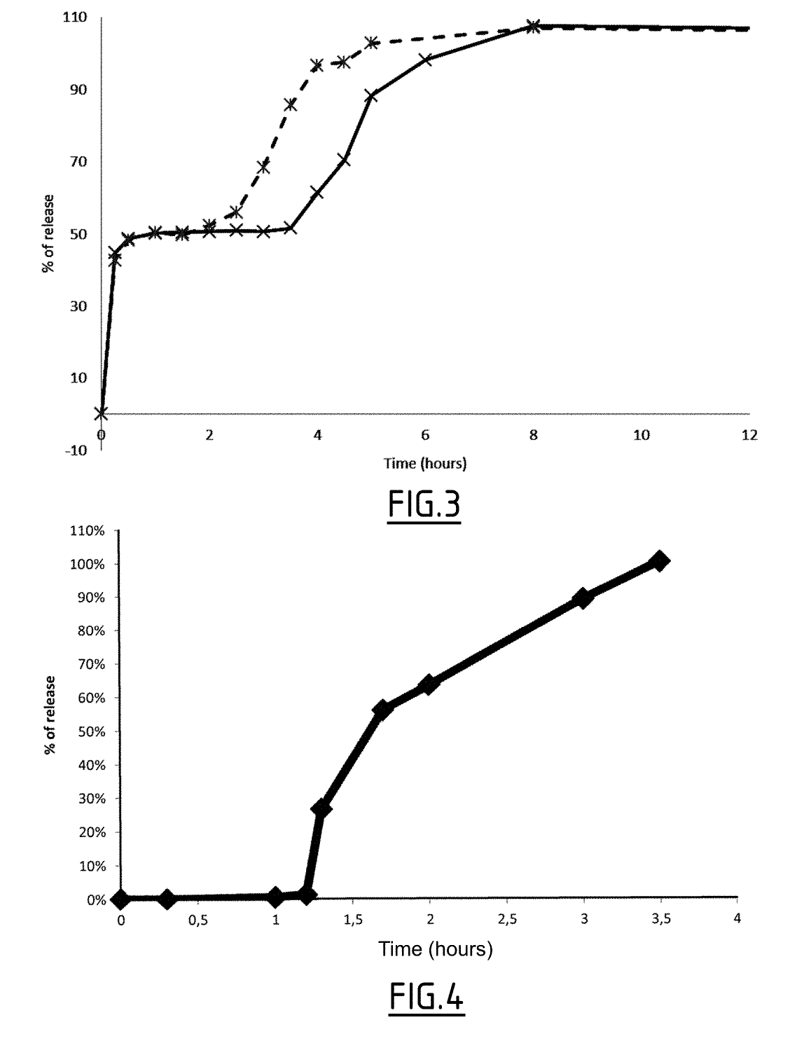

As shown in FIG. 3, a mixture of 50% immediate release (IR) naftazone microspheres and 50% coated naftazone microspheres with controlled release (CR) shows a first fast release, followed by a lag time of two hours or four hours for the different coating level of Surelease before second pulsed release.

This result shows that the combination of IR naftazone microspheres and CR naftazone microspheres allows a bi-pulse release of naftazone.

### Example 4: Preparation of Controlled-Release Naftazone Microspheres (CR) with pH-Dependent (Superior to pH 7) Coating Layer

Starting from the microspheres of example 1, a coating is carried out by using the layer (pH-dependent layer) containing:

| Ingredients | Amount (g) |
|---|---|
| Eudragit ® FS 30D | 66.7 |
| PlasAcryl T20 | 3 |
| Purified water (removed during processing) | 30.3 |
| Total | 100 |

100 g of naftazone microspheres are placed into a fluidized bed (Mini Glatt) with wurster and pre-heated to 35° C. During processing, coating conditions are maintained for temperature product (32° C.), spray rate (1.3 g/min), air flow (27 m³/h) and spray pressure (1.5 b).

Dissolution Test

In order to estimate the characteristic of naftazone release of coated microspheres described in example 4, a dissolution test was performed under following condition:

| | |
|---|---|
| Apparatus | Apparatus 1 (basket) conforming to |
| | Pharmacopeias (for example USP <711>) |
| Dissolution Medium | Phosphate buffer pH 6.8 and pH 7.3 |
| Dissolution volume | 2,000 mL |
| Basket rotation speed | 75 rpm |
| Sampling time point (hours) | 0.3, 1.0, 1.2, 1.3, 1.7, 2.0, 3.0 and 3.5 |
| Bath temperature | 37.0° C. (±0.5° C.) |

Dissolution Analysis Method
Using a graduated cylinder, measure and introduce 2,000 mL of dissolution media (pH 6.8 and pH 7.3) into each dissolution vessel
Equilibrate filled vessels to 37° C.±0.5° C.
Introduce the basket containing the quantity of coated microspheres corresponding to equivalent of 5 mg naftazone into dissolution vessel at pH 6.8 firstly and stir at 75 rpm. After 1 hour, the basket was transferred to dissolution vessel containing pH 7.3 buffer medium.
At each sampling time, withdraw about 2 mL of solution from each vessel, filter and dilute with sample solvent (water/acetonitrile, 1/1:v/v)
Analyze diluted samples using HPLC/UV method

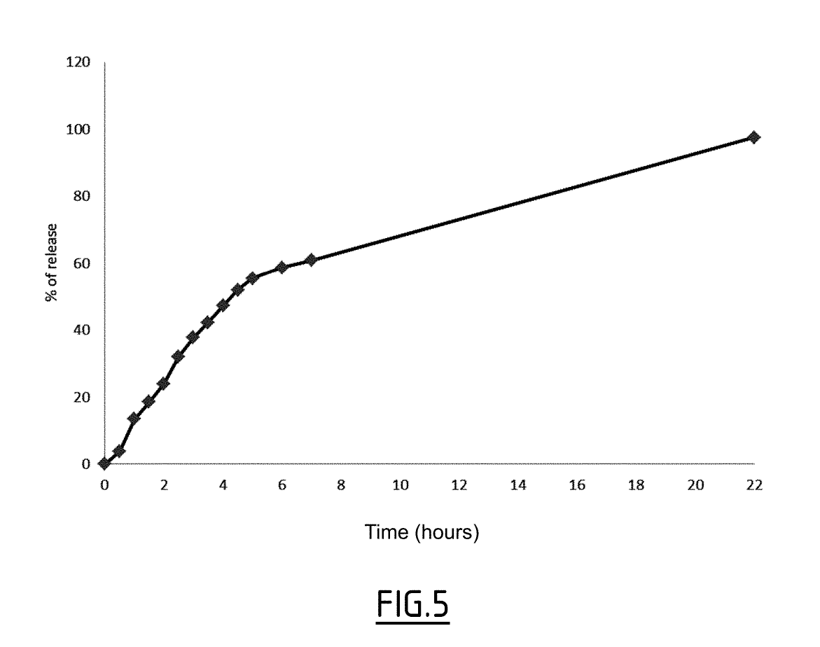

As shown in FIG. 4 concerning the results of the dissolution test of the CR pH-dependent microspheres, there is no dissolution occurred in pH 6.8 medium. Once the microspheres transferred to pH 7.3 medium, the dissolution of naftazone occurs rapidly.

### Example 5 (Comparative): Dissolution Test for One Coated Layer Naftazone Microspheres

The dissolution test was also carried out for naftazone microspheres with only one coating layer of Surelease as explained above in example 2.

As shown in FIG. 5, without the first swelling layer, the dissolution of coated naftazone microspheres with only surelease layer showed a classical sustained release deprived of pulse release.

### Example 6 (Comparative): Comparison with a Sustained-Release (SR) Formulation

The example of naftazone capsules SR filled with different particle size of granules prepared using melt & mix method with lipid SR agent glycerol dibehenate (Compritol® 888 ATO), a lipid excipient from Gattefossé, at different ratio is given below.

Preparation of SR Capsules
Heat Compritol® 888 ATO at 100° C.
Disperse naftazone (in different ratio 1/1 or 1/2) into the molten Compritol
Pour the dispersion into a drying tray and leave to completely recrystallize (>12 h)
Break the dispersion into small pieces manually,
Adjust the small pieces to granules through a sieve with a chosen mesh (from 300 μm to 1,200 μm) using ERWEKA FGS Granulator
Fill the granules at desired dose in capsule

Dissolution Testing of SR Capsules
USP Apparatus I: 100 rpm (paddle apparatus)
Dissolution medium: 1,000 mL Sodium Lauryl Sulfate solution at 1.0%
Dissolution bath temperature: 37° C.±0.5° C.
Sampling times (hours): 0.25, 0.5, 1, 2, 4, 8 and 24 h.
UV wavelength for assay: 270 nm

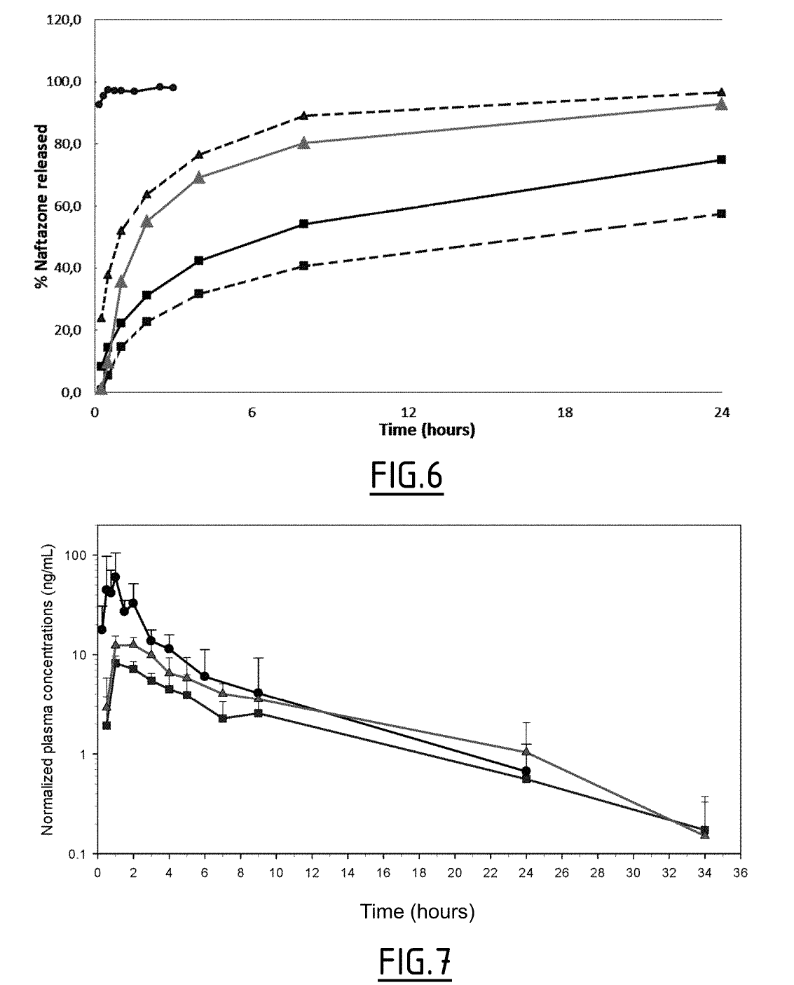

As shown in FIG. 6, dissolution profiles of the capsules filled with the SR granules are well sustained and adjustable from 5 h to over 24 h, fully in agreement with what was expected.

PK Dog of SR Capsules

To confirm the sustained release of naftazone capsule SR based on Compritol in vivo and have in vitro/in vivo correlation (IVIVC), a pharmacokinetic dog study was performed in comparison with naftazone capsule immediate release (IR) formulation.

A PK study was conducted in dog in order to compare the pharmacokinetic (PK) profile and systemic exposure to naftazone after single oral administration in an instant release formulation or in two different sustained release formulations. The tested formulations were:
No 1 (F1): 90 mg of Naftazone (ca. 10 mg/kg) filled in hard gelatin capsule corresponding to immediate release formulation;
No 2 (F2): 810 mg of sustained release grain at size of 800 μm comprising naftazone and Compritol at 1/2 ratio, and 90 mg of Glycolys in hard gelatin capsule eq. to 270 mg of Naftazone (ca. 30 mg/kg);
No 3 (F3): 810 mg of sustained release grain at size of 300 μm comprising naftazone and Compritol at 1/2 ratio, and 90 mg of Glycolys in hard gelatin capsule eq. to 270 mg of Naftazone (ca. 30 mg/kg).

Each formulation was given to 4 fasted dogs. Plasma samples were collected up to 24 h (F1) or 48 h (F2 and F3) post-dosing. Naftazone was determined in plasma samples using an HPLC-MS/MS method. The limit of quantification of the method was 0.5 ng/mL.

To allow direct comparison between formulations, plasma concentrations were normalized to a 1-mg/kg dose, assuming that the naftazone PK is roughly linear in this dose-range. Mean normalized plasma concentration-versus-time profiles and PK parameters of naftazone are presented in FIG. 7.

The PK profiles clearly show that F2 and F3 failed to provide sustained naftazone plasma levels in dogs. The bioavailability of naftazone given in the SR formulations F2 and F3 is significantly lower than in the IR formulation F1 (2.7- and 1.9-fold lower, respectively). In addition, the bioavailability of naftazone given in the SR formulation with the slowest in-vitro dissolution rate (F2) tends to be lower than in the intermediate dissolution rate formulation F3.

1-17. (canceled)

18. A method for the pulsatile release of naftazone or one of its pharmaceutically acceptable salts, pharmaceutical composition comprising naftazone or one of its pharmaceutically acceptable salts, wherein:
a first pulse of naftazone or one of its pharmaceutically acceptable salts is released substantially immediately upon oral administration of said pharmaceutical composition, and
at least one additional pulse of naftazone or one of its pharmaceutically acceptable salts is released at about 3 hours to about 8 hours following said oral administration.

19. A bi-pulsatile release pharmaceutical composition comprising:
at least one immediate-release pharmaceutical system containing naftazone or one of its pharmaceutically acceptable salts, that provides a first pulse of naftazone or one of its pharmaceutically acceptable salts, and
at least one controlled-release pharmaceutical system containing naftazone or one of its pharmaceutically acceptable salts, that provides a second pulse of naftazone or one of its pharmaceutically acceptable salts, wherein:
the first pulse is released substantially immediately upon oral administration,
the second pulse is released at about 3 hours to about 8 hours following oral administration.

20. The pharmaceutical composition of claim 19, wherein the immediate-release pharmaceutical system contains at least 40 mg of naftazone, and wherein the controlled-release pharmaceutical system contains at least 40 mg of naftazone.

21. The pharmaceutical composition of claim 19, wherein the immediate-release pharmaceutical system is made of solid particles of naftazone or of one of its pharmaceutically acceptable salts.

22. The pharmaceutical composition of claim 21, wherein the solid particles are spherical particles the particle size of which being comprised between 500 μm to 1,500 μm.

23. The pharmaceutical composition of claim 22, wherein the spherical particles comprise from 30% to 80% of naftazone or of one of its pharmaceutically acceptable salts, relative to the total weight of said spherical particles.

24. The pharmaceutical composition of claim 22, wherein the spherical particles comprise at least one excipient chosen from the group consisting of: disintegrants, fillers, diluents, plasticizers, surfactants, binders, lubricants, and mixtures thereof.

25. The pharmaceutical composition of claim 22, wherein the spherical particles comprise at least one disintegrant chosen from the group consisting of: croscarmellose sodium, sodium starch glycolate, crospovidone, and mixtures thereof.

26. The pharmaceutical composition of claim 22, wherein the spherical particles comprise at least 0.05% by weight of at least one surfactant, relative to the total weight of said spherical particles.

27. The pharmaceutical composition of claim 21, wherein the spherical particles comprise:
from 30% to 80% by weight of naftazone or of one of its pharmaceutically acceptable salts, relative to the total weight of said spherical particles,
from 5% to 35% by weight of microcrystalline cellulose, relative to the total weight of said spherical particles,
from 5% to 40% by weight of carrageenan, relative to the total weight of said spherical particles, and
from 5% to 30% by weight of sorbitol, relative to the total weight of said spherical particles; and
from 0.05% to 0.25% by weight of sodium lauryl sulfate, relative to the total weight of said spherical particles.

28. The pharmaceutical composition of claim 19, wherein the controlled-release pharmaceutical system is made of coated spherical particles of naftazone or of one of its pharmaceutically acceptable salts.

29. The pharmaceutical composition of claim 28, wherein the coated solid particles comprise a first coating layer containing at least one swelling agent chosen from the group consisting of: croscarmellose sodium, low substituted hydroxypropyl cellulose, sodium starch glycolate, crospovidone, and mixtures thereof, and/or at least one plasticizer chosen from the group consisting of: polyethylene glycol, dibutyl sebacate, phthalate, propylene glycol, triethyl citrate and mixtures thereof.

30. The pharmaceutical composition of claim 28, wherein the coated solid particles comprise one coating layer containing at least one anionic copolymer chosen from the group consisting of: methacrylic acid and an ester chosen from the group consisting of: methyl methacrylate, ethyl acrylate, methyl acrylate, and mixtures thereof, and/or at least one anti tacking or glidant agent.

31. The pharmaceutical composition of claim 28, wherein the coated solid particles comprise a second coating layer containing at least one hydrophobic coating agent.

32. The pharmaceutical composition of claim 28, wherein the coated solid particles comprise one pH-dependent coating layer containing at least one water soluble anionic polymer agent.

33. A method for treating Parkinson disease, comprising the administration of a pharmaceutical acceptable amount of the pharmaceutical composition of claim 18 to a patient in need thereof.

34. The method of claim 33, wherein the pharmaceutical composition is administered once or twice a day.

35. A method for treating Parkinson disease, comprising the administration of a pharmaceutical acceptable amount of the pharmaceutical composition of claim 19 to a patient in need thereof.

36. The method of claim 35, wherein the pharmaceutical composition is administered once or twice a day.

37. The pharmaceutical composition of claim 22, wherein the spherical particles comprise at least 0.05% by weight of at least one surfactant, relative to the total weight of said spherical particles, and wherein the spherical particles comprise from 5% to 35% by weight of at least one plasticizer, relative to the total weight of said spherical particles.
